(19) United States
(12) Patent Application Publication
(10) Pub. No.: US 2024/0197580 A1
(43) Pub. Date: Jun. 20, 2024

(54) MICELLAR BI-PHASE MAKEUP SPRAY IN A NON-AEROSOL FINE MIST PACKAGING

(71) Applicant: L'OREAL, Paris (FR)

(72) Inventors: Samantha SIU, Yorba Linda, CA (US); Paula CZIRYAK, Eatontown, NJ (US); Masanori ORITA, Kawasaki Kanagawa (JP); Sho WATANABE, Kawasaki Kanagawa (JP)

(73) Assignee: L'ORAL, Paris (FR)

(21) Appl. No.: 18/539,467

(22) Filed: Dec. 14, 2023

Related U.S. Application Data
(60) Provisional application No. 63/387,848, filed on Dec. 16, 2022.

(30) Foreign Application Priority Data
Apr. 7, 2023 (FR) 2303507

(51) Int. Cl. A61K 8/02; A61K 8/06; A61K 8/31; A61Q 1/14

(52) U.S. Cl. CPC A61K 8/0291; A61K 8/062; A61K 8/31; A61Q 1/14; A61K 2800/34; A61K 2800/48; A61K 2800/5922; A61K 2800/594; A61K 2800/87

(57) ABSTRACT

A makeup removal composition includes a blend of oils comprising one or more branched or linear liquid alkanes with carbon chain length of C11 to C20, including isohexadecane and isododecane, and one or more emollient esters, at least one polymeric thickener for a water-based system, at least one polyglyceryl nonionic surfactant and water in a bi-phase micellar system having oily and water phases, the water phase including water present from about 30% to about 70% by weight of the makeup removal composition wherein water and oil are present in a ratio of water to oil from about 1.5:1 to about 1:1.5. The product removed by wiping without rinsing with water.

### RELATED APPLICATION

The present application claims priority to U.S. Provisional Application No. 63/387,848 entitled MICELLAR BI-PHASE MAKEUP SPRAY IN A NON-AEROSOL FINE MIST PACKAGING, which was filed on Dec. 16, 2022, and French Application No. FR2303507 filed on Apr. 7, 2023, the entireties of which are incorporated herein by reference.

### FIELD

This invention relates to a makeup removal composition, more specifically a non-aerosol spray micellar bi-phase makeup removal composition.

### BACKGROUND

Removal of stubborn makeup has always been a challenge and a consumer pain point, particularly for waterproof mascara, long-wear foundation and longer-lasting lip products. Typical makeup removal products are ineffective in removing stubborn makeup, thus requiring repeated tugging and rubbing on the skin and/or the delicate eye areas, causing irritation. Some oil-based makeup removing products may provide ease of makeup removal but leave behind an unpleasant and greasy skin feel. In addition, many commercial makeup removal products require application using wipes or pads, and can be messy and result in wasted product due to saturation on pads and dripping.

Spray type products offer a greater ease of application and can provide a more even application followed by manual rubbing without the need for wipes or pads, thus avoiding tugging of sensitive tissues. A key drawback to existing spray formulations is the inclusion of high amounts of oils which can have a greasy feel and leave skin feeling less fresh and clean than is desired by most consumers. Other undesirable components in current makeup removers include alcohols, which can be drying to the skin, as well as sulfate based surfactants, silicones, VOC propellants, and other synthetic components that are less attractive to consumers seeking products that include natural components.

Accordingly, there is a need for a composition that will provide easy and efficacious makeup removal in a spray form that leaves a clean and refreshing skin feel after use without the use of silicone, alcohols and sulfates.

### SUMMARY

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

The disclosure provides, in various embodiments, a non-aerosol spray-on micellar bi-phase makeup removal composition.

In accordance with the various embodiments according to the disclosure, the makeup removal composition includes a blend of oils comprising one or more branched or linear liquid alkanes with carbon chain length of C11 to C20, comprising isohexadecane and isododecane, and one or more emollient esters, at least one polymeric thickener for a water-based system, at least one polyglyceryl nonionic surfactant, and water, in a bi-phase micellar system comprising oily and water phases, the water phase including water present from about 30% to about 70% by weight of the makeup removal composition wherein water and oil are present in a ratio of water to oil from about 1.5:1 to about 1:1.5.

In one embodiment, the disclosure provides a makeup removal composition, comprising: a bi-phase micellar system comprising an oil phase and a water phase, the system comprising:
a. a blend of oils comprising
   i. one or more branched or linear liquid alkanes with carbon chain length of C11 to C20, comprising isohexadecane and isododecane, and
   ii. one or more emollient esters;
b. at least one polymeric thickener for a water-based system;
c. at least one polyglyceryl nonionic surfactant; and
d. water present from about 30% to about 70% by weight of the makeup removal composition
wherein the composition is a micellar bi-phase wherein water and oil are present in a ratio of water to oil from about 1.5:1 to about 1:1.5.

In accordance with the various embodiments, the makeup removal composition is formulated as an aesthetically pleasing two-phase formulation with a phase interface and which allows easy make-up removal that is not greasy, and includes little or no components that may be irritating to the skin or eyes.

In some embodiments, the makeup removal composition is essentially free of silicone.

In some embodiments the composition excludes silicones and one or more of alcohols, sulfate-containing compounds, PEG surfactants, xanthan gum, or combinations thereof.

In various embodiments, the makeup removal composition may further include one or more additives including preservatives, fragrances, actives, and pH adjusters, among other cosmetically acceptable additives.

In some embodiments, the one or more emollient esters comprises isopropyl myristate, and the at least one polymeric thickener for a water-based system comprises hydroxyethylcellulose.

In some embodiments, isohexadecane is present from about 15% to about 30% and isododecane is present from about 10% to about 20%, each by weight, based on the weight of the makeup removal composition.

In some embodiments, isohexadecane is present at about 28% and isododecane is present at about 15%, each by weight, based on the weight of the makeup removal composition.

In some embodiments, the one or more emollient esters is present from about 1% to about 8% by weight, based on the weight of the makeup removal composition.

In some embodiments, the one or more emollient esters comprises isopropyl myristate present from about 1% to about 8% by weight, based on the weight of the makeup removal composition.

In some embodiments, the total amount of emollient ester does not exceed a total of about 10% by weight of the makeup removal composition.

In some embodiments, the at least one polymeric thickener for a water-based system is present from about 0.01% to about 1.0% by weight, based on the weight of the makeup removal composition.

In some embodiments, the at least one polymeric thickener for a water-based system comprises hydroxyethylcellulose.

In some embodiments, the at least one polyglyceryl nonionic surfactant is present in a total amount from about 0.01% to about 0.2%, by weight, based on the weight of the composition.

In some embodiments, the at least one polyglyceryl nonionic surfactant comprises Polyglyceryl-6 Caprylate and Polyglyceryl Dicaprate.

In some embodiments, the at least one polyglyceryl nonionic surfactant comprises polyglyceryl-6 Caprylate present at about 0.025% and Polyglyceryl Dicaprate present at about 0.025%, each by weight, based on the weight of the composition.

In some embodiments, the makeup removal composition comprises triethyl citrate.

In some embodiments, the makeup removal composition includes one or a combination of additives selected from sodium hydroxide, panthenol, hydroxyacetophenone, gluconolactone, myrtrimonium bromide, sodium citrate, sodium chloride, dicaprylyl ether, citric acid, or combinations thereof.

In some embodiments, the disclosure provides a makeup removal composition, comprising: a bi-phase micellar system comprising an oil phase and a water phase, the system comprising:
a. a blend of oils comprising
   i. one or more branched or linear liquid alkanes with carbon chain length of C11 to C20, comprising isohexadecane present at about 28%, and isododecane present at about 15%, and
   ii. one or more emollient esters comprising isopropyl myristate present at about 1%;
b. at least one polymeric thickener for a water-based system comprising hydroxyethylcellulose present at about 0.025%;
c. at least one polyglyceryl nonionic surfactant comprising Polyglyceryl-6 Caprylate present at about 0.025% and Polyglyceryl Dicaprate present at about 0.025%;
d. water present from about 45% to about 55%;
e. triethyl citrate; and
f. one or a combination of additives selected from sodium hydroxide, panthenol, hydroxyacetophenone, gluconolactone, myrtrimonium bromide, sodium citrate, sodium chloride, dicaprylyl ether, citric acid, or combinations thereof, all amounts by weight, based on the weight of the makeup removal composition
wherein the composition is a micellar bi-phase wherein water and oil are present in a ratio of water to oil of about 1:1.

In some embodiments, the disclosure provides a product comprising: a container comprising the makeup removal composition according to the present invention; an outlet connected to the container, such as an outlet of a spray nozzle; and a spray-device, wherein the spray-device is capable of discharging the makeup removal composition in the container via the outlet, wherein the spray nozzle is a fine mist nozzle that delivers a mist having a pump volume of about 0.02 ml (in a range from about 0.01 to about 1.0 ml/pump), and having particle (droplets) of micron scale in a range from about 10 microns to about 900 microns, or from about 40 microns to about 300 microns, or of average size of about 80 to about 100 microns. In some such embodiments, the makeup removal composition as contained in the container is transparent or translucent, wherein the term “translucent” refers to a property of a material which allows light to pass through without making it possible to distinguish alphanumeric characters using 5 mm thick samples, and the term “transparent” refers to a property of a material which allows light to pass through, and makes it possible to distinguish alphanumeric characters using 5 mm thick samples. In some particular embodiments, the mist droplets are also translucent or transparent.

The makeup removal composition may be applied for use and removed by wiping without rinsing with water.

These and other aspects of the invention are set out in the appended claims, and described in greater detail in the detailed description of the invention.

It is to be understood that both the foregoing general description and the following detailed description are exemplary and explanatory only and are not restrictive of the disclosure.

### BRIEF DESCRIPTION OF THE DRAWINGS

Features and advantages of the general inventive concepts will become apparent from the following detailed description made with reference to the accompanying drawings.

FIG. 1 is a chart demonstrating tested features of the inventive makeup remover composition;

FIG. 2 is a chart demonstrating tested features of the inventive makeup remover composition;

FIG. 3 is a chart demonstrating the clinical testing experience of subjects with the inventive makeup remover composition and comparative composition.

FIG. 4 is a chart demonstrating the clinical testing experience of subjects using a misting pump vs an aerosol spray with the inventive makeup remover composition and comparative composition, respectively.

### DETAILED DESCRIPTION

“Keratinous substrate” and “keratinous tissue” each includes but is not limited to skin, hair, and nails.

“Cosmetically acceptable” means a carrier that is compatible with any keratinous substrate.

“Silicone-free” means containing less than 5%, or less than 4%, or less than 3%, or less than 2%, or less than 1%, or less than 0.1% by weight, based on the total weight of the makeup removal composition, of silicones, such as silicone polymers, for example selected from dimethicone and other silicone oils, and silicon elastomers.

“Stability” is evidenced by one or both of unaided visual or microscopic inspection. For purposes hereof, a composition that demonstrates physical stability and does not demonstrate phase instability, and maintains two stable phases that are mixed essentially only by agitation. In various embodiments, an inventive composition remains stable at temperatures in the range from about 4° C. to about 45° C., over a time period of at least one month, or at least two months, or longer, or any value, range, or sub-range therebetween. In some embodiments, a makeup removal composition is stable at 25° C. for at least one month, or at least two months, or at least three months, or longer, or any value, range, or sub-range therebetween. In some embodiments, a makeup removal composition is stable at 37° C. for at least one month, or at least two months, or at least three months, or longer, or any value, range, or sub-range therebetween. As disclosed and exemplified herein, a makeup removal composition is stable at 4° C. for at least one month, or at least two months, or at least three months, or longer, or any value, range, or sub-range therebetween. And as disclosed and exemplified herein, a makeup removal composition is stable through at least 10 freeze thaw cycles.

According to the disclosure, the inventive compositions overcome the shortcomings of the prior art and provide unexpected benefits that include ease with overall makeup removal, easy foundation removal, leaves skin feeling clean and fresh, soft and smooth, easy mascara removal (regular and waterproof) with minimal rubbing, and does not leave a greasy feel or residue.

In some embodiments, the makeup removal composition is a silicone-free, alcohol-free bi-phase makeup remover in a spray format product that is non-aerosol and gives a fine mist, wherein the water phase is confirmed to be micellar technology, and the ingredients are primarily natural or nature based. In some embodiments, the makeup remover composition is a bi-phase with 50% Oil and 50% Water (by mass).

In various embodiments, the makeup removal composition is shaken or agitated to form an emulsion just prior to application. In some embodiments, the inventive makeup removal composition demonstrates phase separation after shaking. In some embodiments, when agitated, the makeup removal composition forms a milky single phase which separates back into three or more visibly distinct phases in a maximum period of 24 hours. In some embodiments, the inventive makeup removal composition demonstrates phase separation after shaking wherein after agitation is ceased, demonstrates slow initial phase separation to enable sufficient homogenous emulsion before dispensing. In some embodiments, the inventive makeup removal composition demonstrates after agitation is ceased and there is a clear container wall with minimal liquid droplet visible to the eye wherein there are essentially no clinging or dragging of a layer on a container wall and there are no or minimal liquid droplets visible at any phase interface. In some embodiments, the makeup removal composition demonstrates excellent ability to remove both long wear mascara and long wear foundation.

Spray delivery of the makeup removal composition allows for more contact time of the composition on skin and therefore, removes the makeup most effectively. Optimized oil and water ratio in the range as disclosed herein and as exemplified provide ideal makeup removal efficacy for all types of makeup. The optimized level of water as disclosed herein and as exemplified eliminates residue on skin, particularly as compared with anhydrous, high oil, and silicone oil containing compositions. And the optimized combination and composition of different chain lengths of hydrocarbons in the oil phase provide ideal makeup removal efficacy particularly for waterproof makeup, most particularly waterproof mascara.

As compared with benchmark products that include propellants, silicones, alcohols, and other less desirable ingredients, the makeup remover composition according to the disclosure differs from the benchmark by straying from aerosol packaging to dispense product as a fine mist without the use of aerosolizing propellants, providing a pleasant mist and enhanced stability. And lacking silicone oils, the makeup removal composition resolves the greasy and oily after-use feel, which is a major detractor of competitive products. In addition, the makeup removal composition is free of harsh chemicals (silicones, alcohol, paraben, fragrance, sulfates). The spray format of the makeup removal composition is most effective at makeup removal when compared to a traditional micellar without spray. And the biphasic nature removes all types of makeup, including long wear mascara, in a single swipe. In addition, the makeup removal composition's water phase is a micellar that demonstrates loosening of stubborn makeup without a greasy residue as compared to the 100% anhydrous competitive formulation, as exemplified herein, and further boosts cleansing capability. The makeup removal composition is mild to the skin and non-irritating to skin and eye and provides a clean skin finish to reduce the need to rinse after application, which in turn, decreases water usage.

Through expert evaluations of makeup removal efficacy, the makeup removal composition according to the disclosure has demonstrated performance on par with competitor anhydrous silicone containing composition when removing foundation and excels at removing eye and lip makeup. Consumer tests also show that the makeup removal composition is favored over the competitor benchmark and does not leave behind any greasy residue. And the makeup removal composition is also ranked high efficiency for mascara removal, and is mild to skin. In vitro tests demonstrate that the composition does not adversely affect the skin condition, and the osmolality data together with the clinical testing show that the makeup removal composition is non-irritating to eyes.

In accordance with the various embodiments according to the disclosure, the makeup removal composition includes a blend of oils comprising one or more branched or linear liquid alkanes with carbon chain length of C11 to C20, comprising isohexadecane and isododecane, and one or more emollient esters, at least one polymeric thickener for a water-based system, at least one polyglyceryl nonionic surfactant and water in a bi-phase micellar system comprising oily and water phases, the water phase including water present from about 30% to about 70% by weight of the makeup removal composition wherein water and oil are present in a ratio of water to oil from about 1.5:1 to about 1:1.5. Removal of the composition may be accomplished by wiping without the need for rinsing or washing as is typically required with oil and surfactant laden compositions such as the benchmark.

### (i) Branched or Linear Liquid Alkane

In accordance with the disclosure, the makeup removal composition includes at least one branched or linear liquid alkane with carbon chain length of C11 to C20. In various embodiments, liquid alkanes may be selected from those with a carbon chain length of from C11 to C20. The liquid alkanes may be selected from those with a carbon chain length of from C11 to C20, or from C15 to C19, or one of C11, C12, C13, C14, C15, C16, C17, C18 to C19. In some particular embodiments, suitable liquid alkanes that may be used according to the disclosure include hydrocarbon-based oils containing from 8 to 16 carbon atoms, and especially branched C8-C16 alkanes such as C8-C16 isoalkanes. In some exemplary embodiments, such liquid alkanes may be chosen from isoparaffins, for instance isododecane (also known as 2,2,4,4,6-pentamethylheptane), isodecane, and isohexadecane.

In some embodiments, the least one branched or linear liquid alkane comprises isohexadecane, C15-19 alkane, isododecane, undecane, tridecane and combinations thereof. In some embodiments the makeup removal composition comprises two or more branched or linear liquid alkanes.

In some embodiments, the branched or linear liquid alkane comprises isohexadecane and isododecane.

In some embodiments, isohexadecane is present from about 15% to about 30% and isododecane is present from about 10% to about 20%, each by weight, based on the weight of the makeup removal composition.

In some embodiments, isohexadecane is present at about 28% and isododecane is present at about 15%, each by weight, based on the weight of the makeup removal composition.

The amount of each of the at least one branched or linear liquid alkane is present in the makeup removal composition in a range of from about 1% to about 35% by weight, or from about 15% to about 30% by weight, or from about 10% to about 20% or any suitable combination, sub-combination, range, or sub-range thereof by weight, based on the weight of the makeup removal composition. One of ordinary skill in the art, however, will appreciate that other ranges are within the scope of the invention.

Thus, each of the at least one branched or linear liquid alkane in the makeup removal composition may be present by weight, based on the total weight of the makeup removal composition, from about 1, 2, 3, 4, 5, 6, 7, 8, 9, 10, 11, 12, 13, 14, 15, 16, 17, 128, 19, 20, 21, 22, 23, 24, 25, 26, 27, 28, 29, 30, 31, 32, 33, 34, to about 35 percent, including increments and ranges therein and there between.

### (ii) Emollient Ester

In accordance with the disclosure, the makeup removal composition includes at least one emollient ester, in some embodiments a branched, alkylated alcohol ester.

In some embodiments, suitable emollient esters that may be used according to the disclosure may be chosen from isopropyl myristate, isopropyl palmitate, ethylhexyl palmitate, methyl palmitate, 2-ethylhexyl palmitate, hexyl laurate, ethylhexyl laurate, isopropyl laurate, ethylhexyl oleate, ethylhexyl isononanoate, isononyl isononanoate, myristyl myristate, 2-ethylhexyl caprate/caprylate (or octyl caprate/caprylate), isostearyl neopentanoate, cetearyl ethylhexanoate, isopropyl isostearate, diisopropyl sebacate, coco caprylate/caprate, diisopropyl adipate, and combinations thereof esters of lactic acid and of fatty alcohols comprising 12 or 13 carbon atoms, and combinations thereof.

In some particular embodiments the at least one emollient ester includes isopropyl myristate.

In some embodiments, the one or more emollient esters is present from about 1% to about 8% by weight, based on the weight of the makeup removal composition.

In some embodiments, the one or more emollient esters comprises isopropyl myristate present from about 1% to about 8% by weight, based on the weight of the makeup removal composition.

In some embodiments, the total amount of emollient ester does not exceed a total of about 10% by weight of the makeup removal composition.

In some embodiments the makeup removal composition comprises two or more emollient esters. In some such embodiments, the at least one emollient ester comprises isopropyl myristate and one or more additional emollient ester. In some particular embodiments, the makeup removal composition comprises isopropyl myristate, isopropyl palmitate, and ethylhexyl palmitate.

In accordance with the various embodiments, each of the at least one emollient esters is present from about 0.1% to about 15% by weight of the makeup removal composition, or from about 1% to about 10% by weight, or from about 1% to about 8%, or about 1% or any suitable combination, sub-combination, range, or sub-range thereof by weight, based on the weight of the makeup removal composition. The total amount of emollient ester present in the makeup removal composition is in a range of from about 0.1% to about 15% by weight, or from about 1% to about 8% by weight, or any suitable combination, sub-combination, range, or sub-range thereof by weight, based on the weight of the makeup removal composition. One of ordinary skill in the art, however, will appreciate that other ranges are within the scope of the invention.

Thus, each of the at least one emollient ester in the makeup removal composition may be present by weight, based on the total weight of the makeup removal composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14 to about 15 percent, including increments and ranges therein and there between

(iii) Polymer

In various embodiments, the makeup removal composition comprises at least one polymer. In some embodiments the at least one polymer is selected from the group consisting of celluloses, gums, hydrophilic colloids, and combinations thereof.

Polymers may be selected from the group consisting of nonionic, anionic, cationic, and amphoteric polymers, such as polysaccharides that include chitosan, chitin, starches, alginates, celluloses, galactomannans such as guar gums and its derivatives, including hydroxypropyl guar, cationic guar derivatives, gums of microbial origin, including xanthan gum, scleroglucan gum, mucopolysaccharides, chondroitin sulfates, and mixtures thereof, and gums derived from plant exudates, including locust bean gums, gum arabic, ghatti gum, karaya gum, gum tragacanth, carrageenan gum, agar gum and carob gum, cellulose-based polymers, including methylcellulose, hydroxyalkylcellulose, ethylhydroxyethylcelluloses, hydroxyethylcellulose, hydroxypropylcellulose, carboxymethylcellulose, cationic cellulose ether derivatives, quaternized cellulose derivatives, nitrocellulose, hemicellulose and hemicellulose derivatives, mannans, xylans, lignins, arabans, galacturonans, alginate-based compounds, glucuronoxylans, arabinoxylans, xyloglucans, glucomannans, fructosans such as inulin, pectic acids and pectins, arabinogalactans, agars, glycosaminoglycans, gelatin, gellan, crosslinked homopolymers of acrylic acid, and combinations thereof.

In some particular embodiments, the makeup removal composition comprises at least one polymer comprising hydroxyethylcellulose. In some embodiments, the makeup removal composition excludes thickeners comprising xanthan gum.

In some embodiments, the at least one polymeric thickener for a water-based system is present from about 0.01% to about 1.0% by weight, based on the weight of the makeup removal composition.

In some embodiments, the at least one polymeric thickener for a water-based system comprises hydroxyethylcellulose.

The at least one polymer present in the makeup removal composition according to the disclosure from about 0.01% to about 10% by weight, or from about 0.01% to about 5% by weight, or from about 0.01% to about 1%, or from about 0.0% to about 0.03%, or about 0.025%, or any suitable combination, sub-combination, range, or sub-range thereof by weight, based on the weight of the makeup removal composition.

Thus, one or a combination of polymers may be present, by weight, based on the total weight of the makeup removal composition, from about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 2, 3, 4, 5, 6, 7, 8, 9, to about 10 weight percent, including increments and ranges therein and there between.

### (iv) Polyglyceryl Nonionic Surfactant

In accordance with the various embodiments, the makeup removal composition includes at least one polyglyceryl nonionic surfactant. In some embodiments, the at least one nonionic surfactant may be selected from polyglyceryl nonionic surfactants are selected from the group consisting of polyglyceryl esters, polyglyceryl ethers, polyglyceryl sorbitan fatty acid esters, combinations of two or more thereof and the like. In some embodiments, the at least one nonionic surfactant is selected from polyglyceryl esters, polyglyceryl ethers, and combinations of two or more thereof.

In some embodiments, the at least one nonionic surfactant is selected from polyglyceryl nonionic surfactants selected from the group consisting of: polyglyceryl-4 caprylate/caprate, polyglyceryl-5 caprylate/caprate, polyglyceryl-6 caprylate/caprate, polyglyceryl-7 caprylate/caprate, polyglyceryl-8 caprylate/caprate, polyglyceryl-9 caprylate/caprate, polyglyceryl-10 caprylate/caprate, polyglyceryl-4 caprate, polyglyceryl-5 caprate, polyglyceryl-6 caprate, polyglyceryl-7 caprate, polyglyceryl-8 caprate, polyglyceryl-9 caprate, polyglyceryl-10 caprate, polyglyceryl-4 laurate, polyglyceryl-5 laurate, polyglyceryl-6 laurate, polyglyceryl-7 laurate, polyglyceryl-8 laurate, polyglyceryl-9 laurate, polyglyceryl-10 laurate, polyglyceryl-6 cocoate, polyglyceryl-7 cocoate, polyglyceryl-8 cocoate, polyglyceryl-9 cocoate, polyglyceryl-10 cocoate, polyglyceryl-11 cocoate, polyglyceryl-12 cocoate, polyglyceryl-6 myristate, polyglyceryl-7 myristate, polyglyceryl-8 myristate, polyglyceryl-9 myristate, polyglyceryl-10 myristate, polyglyceryl-11 myristate, polyglyceryl-12 myristate, polyglyceryl-10 oleate, polyglyceryl-11 oleate, polyglyceryl-12 oleate, polyglyceryl-10 stearate, polyglyceryl-11 stearate, polyglyceryl-12 stearate, and combinations of two or more thereof.

In some embodiments, the at least one nonionic surfactant is selected from polyglyceryl-2 oleate, polyglyceryl-4 caprylate/caprate, polyglyceryl-5 caprylate/caprate, polyglyceryl-6 dicaprate, polyglyceryl-6 caprylate/caprate, polyglyceryl-7 caprylate/caprate, polyglyceryl-8 caprylate/caprate, polyglyceryl-9 caprylate/caprate, polyglyceryl-10 caprylate/caprate, polyglyceryl-4 caprate, polyglyceryl-5 caprate, polyglyceryl-6 caprate, polyglyceryl-7 caprate, polyglyceryl-8 caprate, polyglyceryl-9 caprate, polyglyceryl-10 caprate, polyglyceryl-4 laurate, polyglyceryl-5 laurate, polyglyceryl-6 laurate, polyglyceryl-7 laurate, polyglyceryl-8 laurate, polyglyceryl-9 laurate, polyglyceryl-10 laurate, polyglyceryl-6 cocoate, polyglyceryl-7 cocoate, polyglyceryl-8 cocoate, polyglyceryl-9 cocoate, polyglyceryl-10 cocoate, polyglyceryl-11 cocoate, polyglyceryl-12 cocoate, polyglyceryl-6 myristate, polyglyceryl-7 myristate, polyglyceryl-8 myristate, polyglyceryl-9 myristate, polyglyceryl-10 myristate, polyglyceryl-11 myristate, polyglyceryl-12 myristate, polyglyceryl-10 oleate, polyglyceryl-11 oleate, polyglyceryl-12 oleate, polyglyceryl-10 stearate, polyglyceryl-11 stearate, polyglyceryl-12 stearate, and combinations of two or more thereof.

In some embodiments the makeup removal composition comprises one nonionic surfactant. In some embodiments the makeup removal composition comprises at least two or more nonionic surfactants.

In some embodiments, the makeup removal composition comprises, Polyglyceryl-6 Caprylate and Polyglyceryl Dicaprate.

In some embodiments, the at least one polyglyceryl nonionic surfactant is present in a total amount from about 0.01% to about 0.2%, by weight, based on the weight of the composition.

In some embodiments, the at least one polyglyceryl nonionic surfactant comprises polyglyceryl-6 Caprylate present at about 0.025% and Polyglyceryl Dicaprate present at about 0.025%, each by weight, based on the weight of the composition.

In some embodiments, each of the at least one nonionic surfactant in the makeup removal composition is present from about 0.01% to about 1%, or from about 0.01% to about 0.2%, or from about 0.02% to about 0.03%, or any suitable combination, sub-combination, range, or sub-range thereof by weight, based on the weight of the makeup removal composition. One of ordinary skill in the art, however, will appreciate that other ranges are within the scope of the invention.

Thus, each of the at least one nonionic surfactant is present by weight, based on the total weight of the makeup removal composition, from about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, to about 1.0 percent, including increments and ranges therein and there between.

### (v) Water

In accordance with the various embodiments, water is present in the makeup removal composition in a range from about 30% to about 70%, or from about 35% to about 65%, or from about 40% to about 60%, or from about 45% to about 55%, or about 50%, or any suitable combination, sub-combination, range, or sub-range thereof by weight, based on the weight of the makeup removal composition. One of ordinary skill in the art, however, will appreciate that other ranges are within the scope of the invention. Thus, water is present, by weight, based on the total weight of the makeup removal composition, from about 30, 35, 40, 45, 50, 55, 60, 65, to about 70 weight percent, including increments and ranges therein and there between.

The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

The pH of the makeup removal composition is not limited but is generally from 2 and 12, and in some embodiments, is one of from 3 and 11, and from 5 and 9, and from 6 and 8, and in some embodiments is 7. In some particular embodiments, the pH of the makeup removal composition is in the range from about pH 4 to about pH 7. The pH can be adjusted to the desired value by addition of a base (organic or inorganic) to the makeup removal composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid.

In some embodiments, the makeup removal composition excludes alcohols, for example, monoalcohols such as monohydric C1-C8 alcohols such as ethanol, propanol, butanol, isopropanol, isobutanol, and benzyl alcohol, and phenylethyl alcohol.

### Optional Components

In some embodiments, there may be one or more actives present in the cosmetic composition, according to the disclosure, the additive selected from, for example, preservatives/anti-microbials (for example, chlorphenesin, salicylic acid, phenoxyethanol, potassium sorbate, and caprylyl glycol); actives (for example, hydroxyacetophenone); vitamins (for example, vitamin A, beta carotene, tocopherol/vitamin E, panthenol, retinol, resveratrol, vitamin C, niacinamide, derivatives thereof, and combinations thereof); hyaluronic acid in the form of hydrolyzed hyaluronic acid or sodium hyaluronate; coloring materials/pigments; essential oils; antioxidants(phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria Baicalensis root extract), pine bark extract (Pinus pinaster bark/bud extract), ellagic acid); vitamins and vitamin derivatives, such as calcium pantothenate, tocopherol and ascorbic acid; hydroxy acids; citric acid, sodium citrate, sodium chloride; neutralizing, chelating or pH-adjusting agents (for example, triethylamine (TEA), trisodium ethylenediamine disuccinate, EDTA, and sodium hydroxide); powders, fragrances, dyes, pigments; organic or mineral UV filters; and combinations thereof.

In some particular embodiments, a composition according to the disclosure may include one or a combination of additives selected from sodium hydroxide, panthenol, hydroxyacetophenone, gluconolactone, myrtrimonium bromide, sodium citrate, sodium chloride, dicaprylyl ether, citric acid, or combinations thereof.

Although the aforementioned optional components are given as an example, it will be appreciated that other optional components compatible with cosmetic applications known in the art may be used.

In accordance with the various embodiments, the amount of one or more actives and additives, alone or in combination, when present in the composition according to the disclosure can be present in a range from about 0.0001% to about 20%, and in some embodiments, from about 0.005% to about 0.01%, and in some embodiments, from about 0.01% to about 0.1%, and in some embodiments, from about 0.15% to about 5%, and in some embodiments, from about 0.40% to about 4%, and in some embodiments, from about 0.5% to about 2.5%, and in some embodiments, from about 0.1% to about 0.5% and in some embodiments, from about 1% to about 2%, or any suitable combination, sub-combination, range, or sub-range thereof by weight, based on the total weight of the composition. One of ordinary skill in the art, however, will appreciate that other ranges are within the scope of the invention.

Thus, any one or a combination of actives and additives may be present, each one or the combination present from about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 percent, by weight, including increments and ranges therein and there between.

### Product/Article of Manufacture

The present invention also relates to a cosmetic product comprising the makeup removal composition according to the present invention. The product according to the present invention comprises the makeup removal composition according to the present invention and a container or a package in which the makeup removal composition is contained.

The makeup removal composition of the present invention is sprayable or, in other words, capable of being dispensed in the form of fine particles or a mist. This means that the makeup removal composition is contained and dispensed using a dispenser having a spray nozzle, which in some embodiments is a high definition spray nozzle, capable of delivering a mist having particle (droplets) of micron scale in a range from about 10 microns to about 900 microns, or from about 40 microns to about 300 microns, or of average size of about 80 to about 100 microns.

Accordingly, the present invention also relates to a product comprising a container and a spray nozzle, wherein said container contains the makeup removal composition of the present invention.

The makeup removal composition of the present invention may be applied with any container with spraying means, including suitable aerosol and most preferably non-aerosol spray devices. Therefore, non-aerosol spray devices are preferred, and include for example, mist pumps or spray pumps such as the following: Eurogel Spray Pump (available from Seqquist Perfect, Cary III.), PI spray pump (available from Precision, Ajax, Canada), APTAR oil mist pump, [Rare Beauty] misting pump, Calmar Spray Pumps, Calmar, Inc., City of Industry, Calif, and Mitani Mist Pumps (available from MITANI VALVE CO., LTD).

In one specific embodiment of the present invention, the disclosure provides a product comprising: a container comprising the makeup removal composition according to the present invention; an outlet connected to the container, such as an outlet of a spray nozzle; and a spray-device, wherein the spray-device is capable of discharging the makeup removal composition in the container via the outlet, wherein the spray nozzle is a fine mist nozzle that delivers a mist having a pump volume of about 0.02 ml (in a range from about 0.01 to about 1.0 ml/pump), and having particle (droplets) of micron scale in a range from about 10 microns to about 900 microns, or from about 40 microns to about 300 microns, or of average size of about 80 to about 100 microns. In some such embodiments, the makeup removal composition as contained in the container is transparent or translucent, wherein the term “translucent” refers to a property of a material which allows light to pass through without making it possible to distinguish alphanumeric characters using 5 mm thick samples, and the term “transparent” refers to a property of a material which allows light to pass through, and makes it possible to distinguish alphanumeric characters using 5 mm thick samples. In some particular embodiments, the mist droplets are also translucent or transparent.

The examples below according to the invention are given by way of illustration and without a limiting nature. The names are the chemical name or the INCI name. The amounts are given therein as % by weight, unless otherwise mentioned.

### EXAMPLES

### Example 1: Raw Materials

TABLE 1
Raw Materials
INCI Name

Water
Isohexadecane
Isododecane
Isopropyl Myristate
Polyglyceryl-6 Caprylate
Polyglyceryl Dicaprate
Hydroxyethylcellulose

### Example 2: Inventive Composition

Various representative embodiments of the inventive composition are exemplified herein.

TABLE 2
Inventive Composition Ingredients
Inventive 1
INCI US

ISOPROPYL MYRISTATE*
SODIUM HYDROXIDE
PANTHENOL
WATER
SODIUM CITRATE
HYDROXYETHYLCELLULOSE
TRIETHYL CITRATE*
SODIUM CHLORIDE
ISOHEXADECANE*
ISODODECANE*
DICAPRYLYL ETHER
POLYGLYCERYL-6 DICAPRATE*
HYDROXYACETOPHENONE
POLYGLYCERYL-6 CAPRYLATE*
GLUCONOLACTONE
MYRTRIMONIUM BROMIDE
CITRIC ACID

TABLE 3
Inventive Composition (varied by water/oil ratio), wherein INV 1 (also referred to
as INV 1) has an ~50/50 water to oil ratio, and each of the VAR embodiments have
varied water to oil ratio as exemplified and further as set forth in Example 4.

| INCI US | VAR 8 | VAR 7 | VAR 6 | VAR 5 | VAR 4 | VAR 3 |
|---|---|---|---|---|---|---|
| ISOPROPYL MYRISTATE | 1.800 | 1.600 | 1.400 | 1.200 | 0.800 | 0.600 |
| SODIUM HYDROXIDE | 0.0031 | 0.0062 | 0.0093 | 0.0124 | 0.0186 | 0.0217 |
| PANTHENOL | 0.0135 | 0.027 | 0.0405 | 0.054 | 0.081 | 0.0945 |
| WATER | 9.6754 | 19.3508 | 29.0262 | 38.7016 | 58.0524 | 67.7278 |
| SODIUM CITRATE | 0.011 | 0.022 | 0.033 | 0.044 | 0.066 | 0.077 |
| HYDROXYETHYLCELLULOSE | 0.006 | 0.012 | 0.018 | 0.024 | 0.036 | 0.042 |
| TRIETHYL CITRATE | 2.00 | 2.00 | 2.00 | 2.00 | 2.00 | 2.00 |
| SODIUM CHLORIDE | 0.050 | 0.100 | 0.150 | 0.200 | 0.300 | 0.350 |
| ISOHEXADECANE | 49.410 | 43.920 | 38.430 | 32.940 | 21.960 | 16.470 |
| ISODODECANE | 27.00 | 24.00 | 21.00 | 18.00 | 12.00 | 9.00 |
| DICAPRYLYL ETHER | 9.990 | 8.880 | 7.770 | 6.660 | 4.440 | 3.330 |
| POLYGLYCERYL-6 DICAPRATE | 0.005 | 0.010 | 0.015 | 0.020 | 0.030 | 0.035 |
| HYDROXYACETOPHENONE | 0.005 | 0.010 | 0.015 | 0.020 | 0.030 | 0.035 |
| POLYGLYCERYL-6 CAPRYLATE | 0.005 | 0.010 | 0.015 | 0.020 | 0.030 | 0.035 |
| GLUCONOLACTONE | 0.015 | 0.030 | 0.045 | 0.060 | 0.090 | 0.105 |
| MYRTRIMONIUM BROMIDE | 0.007 | 0.014 | 0.021 | 0.028 | 0.042 | 0.049 |
| CITRIC ACID | 0.004 | 0.008 | 0.012 | 0.016 | 0.024 | 0.028 |

TABLE 4
Inventive Composition (continued; varied by water/oil ratio)

| INCI US | VAR 2 | VAR 1 | INV 1 | VAR 9 | VAR 10 | VAR 11 |
|---|---|---|---|---|---|---|
| ISOPROPYL MYRISTATE | 0.400 | 0.200 | 1.00 | 0.8846 | 11.100 | 10.00 |
| SODIUM HYDROXIDE | 0.0248 | 0.0279 | 0.0155 | 0.01728870 | 0.0155 | 0.0155 |
| PANTHENOL | 0.108 | 0.1215 | 0.0675 | 0.07528950 | 0.0675 | 0.0675 |
| WATER | 77.4032 | 87.0786 | 48.377 | 53.95970580 | 48.377 | 48.377 |
| SODIUM CITRATE | 0.088 | 0.099 | 0.055 | 0.06134700 | 0.055 | 0.055 |
| HYDROXYETHYLCELLULOSE | 0.048 | 0.054 | 0.030 | 0.03346200 | 0.030 | 0.030 |
| TRIETHYL CITRATE | 2.00 | 2.00 | 2.00 | 2.00 | 2.00 | 1.00 |
| SODIUM CHLORIDE | 0.400 | 0.450 | 0.250 | 0.27885000 | 0.250 | 0.250 |
| ISOHEXADECANE | 10.980 | 5.490 | 27.450 | 24.28227000 | 26.800 | 28.900 |
| ISODODECANE | 6.00 | 3.00 | 15.00 | 13.269 | 5.550 | 5.550 |
| DICAPRYLYL ETHER | 2.220 | 1.110 | 5.550 | 4.90953000 | 5.550 | 5.550 |
| POLYGLYCERYL-6 DICAPRATE | 0.040 | 0.045 | 0.025 | 0.02788500 | 0.025 | 0.025 |
| HYDROXYACETOPHENONE | 0.040 | 0.045 | 0.025 | 0.02788500 | 0.025 | 0.025 |
| POLYGLYCERYL-6 CAPRYLATE | 0.040 | 0.045 | 0.025 | 0.02788500 | 0.025 | 0.025 |
| GLUCONOLACTONE | 0.120 | 0.135 | 0.075 | 0.08365500 | 0.075 | 0.075 |
| MYRTRIMONIUM BROMIDE | 0.056 | 0.063 | 0.035 | 0.03903900 | 0.035 | 0.035 |
| CITRIC ACID | 0.032 | 0.036 | 0.020 | 0.02230800 | 0.020 | 0.020 |

### Example 3: Comparative Composition #1

TABLE 5
Comparative Composition
INCI US (AEROSOL)

ETHYL TRISILOXANE
ISODODECANE
CYCLOPENTASILOXANE
CETYL ETHYLHEXANOATE
ISOAMYL COCOATE
ISOPROPYL ISOSTEARATE
ISOSTEARYL ISOSTEARATE
SIMMONDSIA CHINENSIS (JOJOBA) SEED OIL
ROSA RUBIGINOSA SEED OIL
FRAGRANCE/PARFUM

TABLE 6
Comparative Composition #2
INCI US (NOT SPRAY)

AQUA/WATER/EAU
CYCLOPENTASILOXANE
ISOHEXADECANE
SODIUM CHLORIDE
POLOXAMER 184
HEXYLENE GLYCOL
DIPOTASSIUM PHOSPHATE
BENZYL ALCOHOL
POTASSIUM PHOSPHATE
QUATERNIUM-15
BENZALKONIUM CHLORIDE
PARFUM/FRAGRANCE
CITRONELLOL
GERANIOL

TABLE 7
Comparative Composition #3
INCI US (NOT SPRAY)

WATER/AQUA
CYCLOPENTASILOXANE
ISOHEXADECANE
POTASSIUM PHOSPHATE
SODIUM CHLORIDE
HEXYLENE GLYCOL
DIPOTASSIUM PHOSPHATE
DISODIUM EDTA
DECYL GLUCOSIDE
POLYAMINOPROPYL BIGUANIDE

TABLE 8
Comparative Composition #6
INCI US

CYCLOPENTASILOXANE
CAPRYLYL METHICONE
TOCOPHERYL ACETATE

TABLE 9
Comparative Composition #5
INCI US (AEROSOL)

CAPRYLIC/CAPRIC TRIGLYCERIDE
C12-15 ALKYL BENZOATE
BUTANE
ISOBUTANE
PROPANE
TOCOPHERYL ACETATE

TABLE 10
Comparative Composition #7
INCI US

PEG-6 CAPRYLIC/CAPRIC GLYCERIDES
GLYCERIN
SODIUM LAUROYL SARCOSINATE
CAPRYL/CAPRAMIDOPROPYL BETAINE
PHENOXYETHANOL
NIACINAMIDE
UREA
DISODIUM EDTA
CHLORPHENESIN
COCONUT EXTRACT

### Example 4: Demonstration of Spray and Sensorial Properties of Inventive Varying Water and Oil Ratio

TABLE 11
Varying O/W from 10:90 to 90:10 & Testing Jetting of Spray Pattern & Sensorial

| | Var 1 | Var 2 | Var 3 | Var 4 | INV 1 | Var 5 | Var 6 | Var 7 | Var 8 |
|---|---|---|---|---|---|---|---|---|---|
| Actual Wt | 5.02 | 10.01 | 15.26 | 20.02 | 25.08 | 30.01 | 35.08 | 40.03 | 45.27 |
| Oil (g) | | | | | | | | | |
| Actual Wt | 45.08 | 40.19 | 35.06 | 30.02 | 25.06 | 20.03 | 15.28 | 10.02 | 5.19 |
| Water (g) | | | | | | | | | |
| Actual Oil | 10.02% | 19.94% | 30.33% | 40.01% | 50.02% | 59.97% | 69.66% | 79.98% | 89.71% |
| % by Mass | | | | | | | | | |
| Actual Water | 89.98% | 80.06% | 69.67% | 59.99% | 49.98% | 40.03% | 30.34% | 20.02% | 10.29% |
| % by Mass | | | | | | | | | |
| Oil Mass % | 10 | 20 | 30 | 40 | 50 | 60 | 70 | 80 | 90 |
| Water Mass % | 90 | 80 | 70 | 60 | 50 | 40 | 30 | 20 | 10 |
| Spray Pattern | Misting | Mist | little jet, | little jet, | Jet/mist, | jetting | jetting | jetting, | very jet, |
| (in UD Euromist) | is best | | requires | requires | foams a | | | foaming | foaming |
| Remarks | | | priming | priming | little | | | | |
| | | | for mist | for mist | | | | | |
| Sensorial | Very | No | No | no oily | no oily | no oily | oily but | oily | very |
| Remarks | watery | residue | residue | residue | residue, | residue | wipes off | residue | sticky/oily |
| | | | | | takes off | | | | residue |
| | | | | | makeup well | | | | |

### Example 5: Demonstration of Makeup Removal Varying Water and Oil Ratio

TABLE 12
Varying O/W from 10:90 to 90:10 & Testing Mascara & Foundation
Removal (# of COMP 2 Rounds to clean)

| | Var 1 | Var 2 | Var 3 | Var 4 | INV 1 | Var 5 | Var 6 | Var 7 | Var 8 |
|---|---|---|---|---|---|---|---|---|---|
| Oil Mass % | 10 | 20 | 30 | 40 | 50 | 60 | 70 | 80 | 90 |
| Water Mass % | 90 | 80 | 70 | 60 | 50 | 40 | 30 | 20 | 10 |
| Maybelline Sky | 3.25 | 2.25 | 1.5 | 1.5 | 0.5 | 1 | 0.75 | 0.25 | 0.25 |
| High Mascara | | | | | | | | | |
| (Waterproof) | | | | | | | | | |
| # COMP 2 | | | | | | | | | |
| Rounds Residue | | | | | | | | | |
| Maybelline | 1.5 | 1.25 | 1 | 0.75 | 0.25 | 0.25 | 0.1 | 0.25 | 0.1 |
| SuperStay 24 H | | | | | | | | | |
| Foundation | | | | | | | | | |
| # COMP 2 | | | | | | | | | |
| Rounds Residue | | | | | | | | | |

Lab Pilot Study makeup remover Efficacy
Longwear Lipstick (Matte Ink) 1 swatch forward 1 swatch backwards, leave for 5 minutes spray swatch with makeup remover twice, spread test sample over swatch and wipe away
SkyHigh Mascara (WTP) (3 pumps, 5 strokes one way, 3 pumps, 5 strokes another way)
SuperStay 24 H foundation (LW) ½ pump foundation, concealer brush to blend swatch

### Example 6: Stability Demonstration

TABLE 13
Stability Testing

| | | | 8 wks. | 4 wks. | 4 wks. at | 8 wks. at | 4 wks. |
|---|---|---|---|---|---|---|---|
| | | T₀ @ | with 10 | pH at | 25, 45, | 25, 45, | pH at |
| Context | | 25° C. | F-T cycles | 25, 45 | 4, 37 | 4, 37 | 25, 45 |
| Comp 2 Phase - | Comp 2 | Clear liquid; | No | NA | ALL: | ALL: | NA |
| (comparable to oil | (Oil | colorless; | change | | No | No | |
| phase in INV 1) | Composition) | odorless | | | change | change | |
| INV 1 water | INV 1 | Clear liquid; | No | 4.76; | ALL: | @ 25 and 4, | 4.99; |
| phase only | (Water | colorless; | change | 4.5 | No | No change; | 4.34 |
| | Phase only) | odorless | | | change | @ 45 and | |
| | | | | | | 37, cloudy | |
| | | | | | | oil phase | |
| INV 1 (water/oil | INV 1 | Clear liquid | No | NA | ALL: | @ 25 and 4, | NA |
| is 50/50) | | biphase; | change | | No | No change; | |
| | | both phases | | | change | @ 45 and | |
| | | droplets at | | | | 37, cloudy | |
| | | interface; | | | | oil phase | |
| | | colorless; | | | | | |
| | | odorless | | | | | |

### Example 7: Osmolality Testing

TABLE 14
Osmolality

| Tested Formulation | Osmolality (mOsm/l) |
|---|---|
| Var 9 + colorants, fragrance | 315 |
| and 0.02% | |
| hydroxyethylcellulose | |
| Var 9 | Extrapolated to 315 |
| INV 1 | Extrapolated to 315 |

Osmolality of a water phase according to Var 9 was tested where in the water phase further included colorants, fragrance and 0.02% hydroxyethylcellulose. In brief, Equipment used includes an automatic Osmometer: OSK 40BU201 (Ogawa-seiki), and protocol for testing includes preparing composition; shaking 20 times and allow to rest for at least 8 hours (for example, overnight); extract water phase and measure osmolality (unit of score is mOsm/l, number of molecules per liter of test material) with osmometer.

There is thus a close transitive relationship between the tested composition and Var 9 and INV 1 (the latter two vary only in oil/water concentration). It is understood in the art that osmolality can be employed as an indirect means to assess whether a formulation may be irritable to the eyes based on the understanding that the osmotic pressure value of tears is a range of 260-290, and that the closer the osmotic pressure (mOsm/l) of a product is to this range, the gentler it is to the eyes. Accordingly, since the value of 315 mOsm/l it is close to that of tears, it is concluded empirically that the listed formulations are suitable as a makeup remover used around the eye area without causing discomfort. These results are corroborated in the clinical studies which demonstrate that there were no reports of skin or mention of any eye irritation by research subjects. In accordance with the various embodiments, the osmolality is in the range from about 260 to about 350 mOsm/l, and in some embodiments from about 300 to about 330 mOsm/l.

While the consumer test proves that the inventive composition is non irritating to skin and eyes in general, this osmolality test is more specific to show whether it is gentle on the eyes, which is a critical portion of the makeup removal process.

### Example 8: CMC Surface Tension Testing

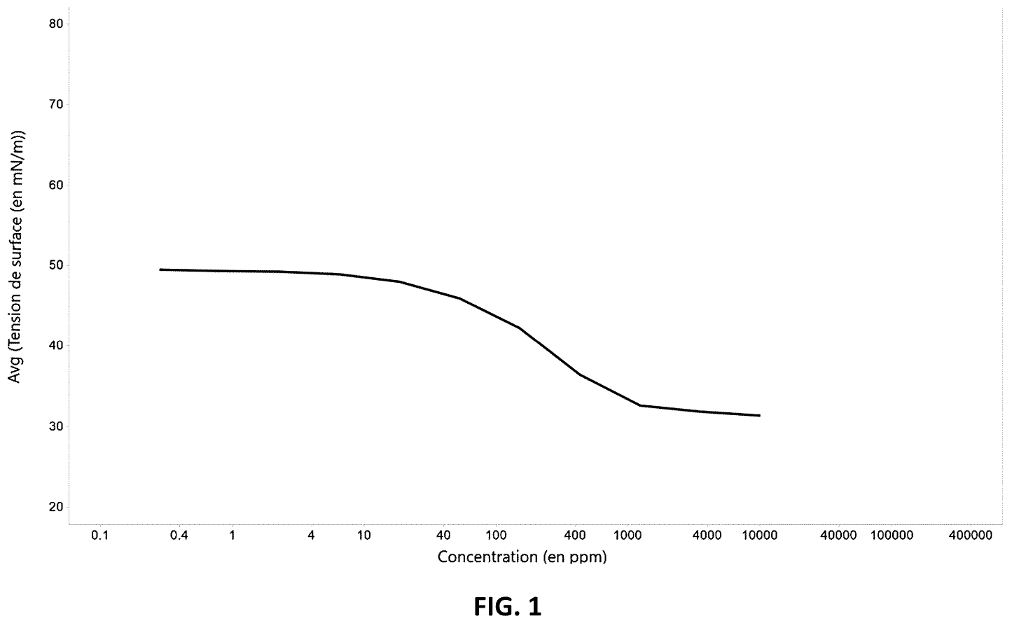

Employing inventive Var 9, Critical micelle concentration was evaluated to demonstrate the concentration of surfactant in a bulk phase, above which aggregates of surfactant molecules, micelles, start to form. Measurements of CMC surface tensioning are performed on a tensiometer. Referring now to FIG. 1, the curve shows decreasing slope of surface tension which plateaus and the surface tension becomes constant. The intersection of the decreasing slope and surface tension plateau is indicative of the presence of micelles. The shape of the curve demonstrates that the water phase of the tested Inventive 1 is micellar.

### Example 9: In Vitro Study: Assessment of Makeup Remover Dissolving Mist Spray Effects on the Skin Barrier Function In Vitro Using Biophysical Tests and Zein Protein Model

The objective of this experiment is to demonstrate the impact of makeup remover on stratum corneum (SC) lipid and Protein and demonstrate the impact of different formula composition i. e. surfactant, humectant etc. on mildness potential of the cleanser.

The study also demonstrates how the inventive composition (INV 1) performs with respect to DSC, TEWL and Zein test.

In-vitro assessment of mildness of makeup remover Dissolving mist spray INV 1 was done. Overall, INV 1 falls into the target mildness zone (roughly delineated by the light green rectangle), where it will neither perturb the lipids (DSC) nor increase the water loss (TEWL).

### Penetration of Water Vapor Through SC (In Vitro TEWL)

The surface of the SC samples was cleaned with isooctane and placed in sample holders of TEWL. The sample holders then immersed in Evian water to wet the SC and dried in ambient atmosphere for at least 1 hour. The surface of the SC sample was pre-wetted with 10 μl per sample, of a solution of a mixture of water and n-propanol (8:2) before being conditioned for 8 hours in a glove box in controlled atmosphere (30° C., 30% RH). Control TEWL measurement “TO” were recorded. SC samples were then immersed in cleanser formulas diluted 4-fold (25%) with the water for 2 hours with gentle stirring on shaker and rinsed by three successive washes of water and the samples were left at room temperature for at least 4 hours to ensure drying. The SC samples were then placed in the temperature and humidity control chamber for equilibrium at 30° C. and 30% RH. After overnight equilibrium, TEWL measurements “T1” were recorded.

Measurement of flow of water crossing a sample of SC situated above a water tank. The measurement was made in a glove box kept at 30° C. and 30% RH by means of a hygrometric probe coupled to a robotic sample holder and a data acquisition system Dermalab. The hygrometric probe contains two humidity sensors located at different heights in the cylindrical probe chamber, so that the humidity gradient can be measured and used to calculate the flux of water vapor. The measurements were performed before and after treatment for each sample so that each sample served its own control to ensure robust measurements.

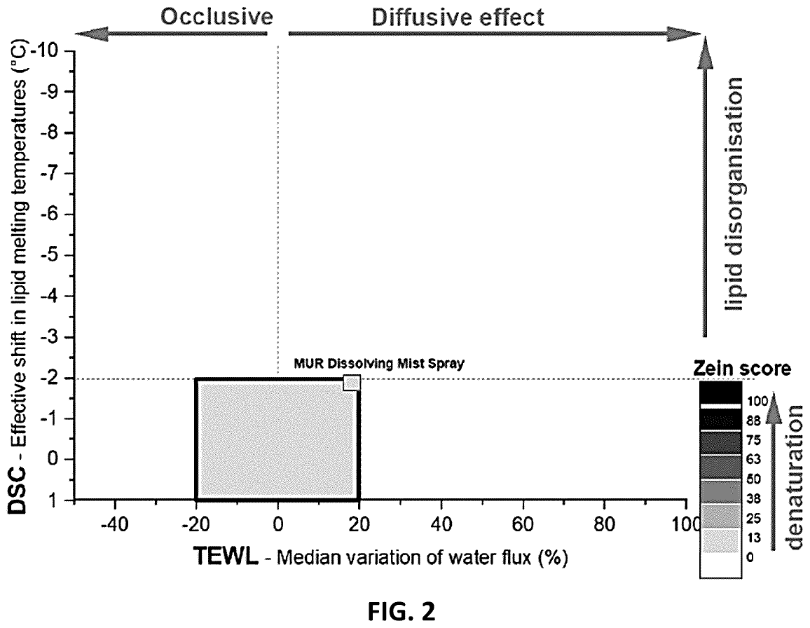

Reference is made to FIG. 2 and Table 15 which provide the results of the tests as shown below. Due to high variability inherent to SC substrate, the results are expressed as a relative variation of TEWL for every sample: Δ(TEWL)=(T1−T0)/T0·100%. Positive variation corresponds to a diffusive effect (weakening of the barrier function), and negative values correspond to an occlusive effect. The mean values were indicated with open squares for reference.

### Assessment of Intercellular SC Lipid Perturbation by DSC

After TEWL measurements, the same samples were used for the DSC analysis after equilibrating them for additional 4 hours at 75% RH, 25° C.

The DSC pan was placed on an analytical balance and the weight was tared. The SC samples were removed from the glove box/temperature and humidity chamber and the samples were removed from sample holders. Two pieces of SC were rolled together and placed in the tared DSC pan and sealed using crimper press. The weight was recorded of DSC pan and subjected for the DSC analysis.

The measurements were performed using aluminum hermetic sealed pans and heating from 10° C. to 110° C. at a rate of 5° C./minute.

Reference is made to FIG. 2 and Table 15 which provide the results of the tests as shown below. A linear combination of the measured shifts for the T2 and T3 transitions for each treatment was used as a measure of the SC lipid disordering following treatment: ΔT_{effective}=0.7·ΔTₘ(T2)+0.3·ΔTₘ(T2). Higher weight was given to the T2 transition as it was our primary criterion of lipid perturbation with well-established interpretation; at the same time, the T3 contribution was also included.

### Assessment of Protein Solubilization by Zein Test

The harshness of surfactants was evaluated by employing the zein solubility test wherein zein, a yellow corn protein, similar to keratin present in the skin, with limited solubility in water is denatured (solubilized) by the surfactant. The ability of surfactants to denature and solubilize zein has been linked to their skin-irritation potential. The soluble zein protein was measured by modified Lowry method using spectrophotometer. The absorption correlates with the solubility of the zein protein which in turn can be considered as a measure of the irritation potential or the harshness of the product. The harshness of the analyzed samples was graded and compared using a 1.5% solution of sodium laureate sulfate (SLS) as a positive control. The harshness data for the samples is expressed in terms of percentage of the SLS score.

The % zein score as calculated against the respective blanks is provided in Table 15, below. The ranking of samples based on the analyzed harshness (% zein score) relative to SLS is illustrated in FIG. 2. Referring now to FIG. 2, the plot shows that shows that the Zein score is a function of the DSC and TEWL score, as explained herein above. Inventive 1 has a Zein score of 48 which falls just under the cusp of strong protein denaturation which falls within the top right of the depicted rectangle which shows it is in the range to be considered a mild formula. A composition according to the disclosure has a Zein scores in a range from about 88 to about 13 which is accomplished by employing surfactants according to the disclosure and avoiding harsh surfactants that would cause lipid disorganization and high Zein solubilization.

### Mapping of Cleansers According to Three Mildness Tests

The results from the three above tests (TEWL, DSC and Zein) can be conveniently combined in a single mapping to visually represent the performance of each formula according to these tree criteria. In the mapping below, the abscissa shows the effect on barrier properties related to water penetration (TEWL) and the ordinata—the effect on the lipid ordering (DSC). The light green rectangle corresponds to the target mildness area, where neither SC permeabilization nor lipid disordering occur. The zein score is represented by the color of the data-points, with red shades indicating a high score (strong protein solubilization/denaturation) and green shades indicating low scores (little protein solubilization/denaturation).

TABLE 15
mildness tests scores

| | | ADSC Lipid | |
|---|---|---|---|
| Sample | ΔPIE Water Flux (%) | Perturbation (C.) | Zein sc. (%) |
| INV 1 | 18 | −1.9 | 48 |

### Example 10: Clinical Studies: Study A

Clinical Studies were conducted to compare the inventive to a commercial benchmark and other commercial spray and non-spray makeup removers.

Clinical Study A: COMP 1, is a continuous mist that slips over skin and instantly dissolves all makeup on contact without leaving skin feeling tight or dry. This no-drip, superfine mist is COMP 1's secret to easily remove full-glam makeup. The formula soothes skin with calming jojoba and brightening rosehip oils and refreshing skin with fragrance notes of green tea, cucumber, and rose. The inventive composition hereof was evaluated vs this competitive benchmark.

Methodology ▪ Blinded, sequential monadic design ▪ 2 products ▪ INV 1▪ COMP 1▪Online recruitment and in person placement ▪ 5 days use with an online survey and video for each product after usage

Recruit Criteria ▪ N=37, Women ▪ Age 18-55 years old ▪ Current users of makeup remover 5 or more days a week—any form ▪ Must not use makeup remover wipes exclusively▪Must be waterproof mascara users (can be dual users, but open to wearing WP for 10 days), and either long wear foundation and/or lipstick ▪ Willing to use a spray makeup remover▪Mass and/or luxury market users ▪ All skin types: Oily, dry, normal, combination ▪ All skin tones▪All ethnicities ▪ Standard security screen

Excluding: ▪ Wipes only user for makeup removal ▪ Allergies or known sensitivities to skin or makeup products ▪ Currently under dermatologist care for face ▪ Very/Extremely sensitive skin and eyes ▪ Prone to severe acne ▪ Facial surgery within the past 3 months▪Pregnant or nursing

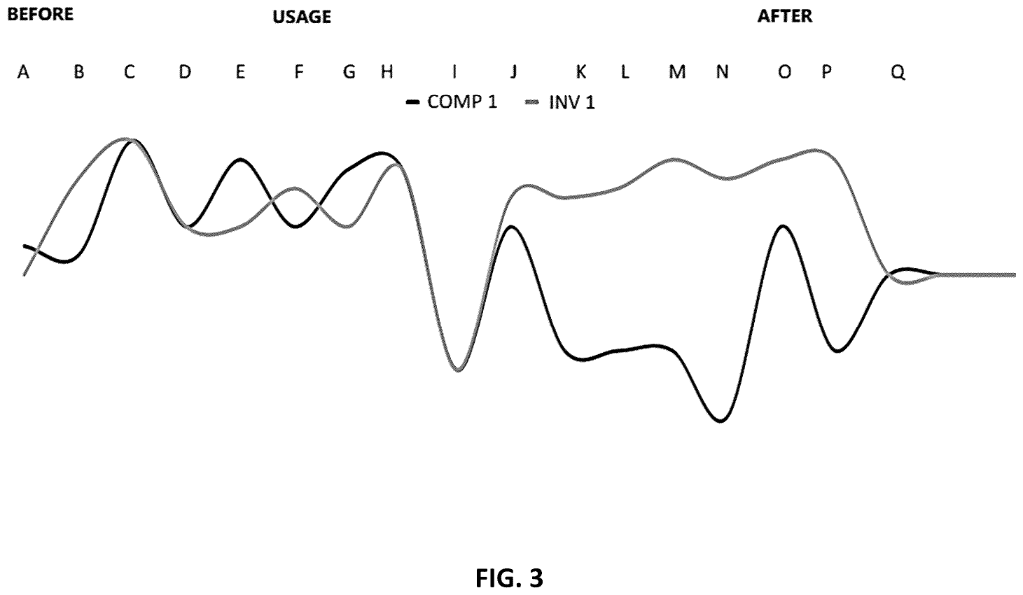

Tested: INV 1 vs COMP 1: Referring to FIG. 3, the chart details the sensory journey of the consumer and reported overall perception according to the tests undertaken in the above described clinical study, where the specific data points are described in Table 16.

Referring again to FIG. 3, noting that these compositions differ significantly in terms of the amount and nature of the oil content where COMP 1 is anhydrous, includes silicone oils/silicones and strong surfactants, INV 1 demonstrates an overall favorable user journey with an opportunity to improve amount of rubbing required for complete removal. COMP 1 had a similar before and during usage journey to the INV 1. However, COMP 1 was less favorable after usage and was less appreciated for leaving skin feeling clean, not leaving residue, and needing to wash face after application, which is unusual.

As shown, INV 1 outperforms COMP 1 in terms of product consistency and shows a large margin of superiority in the after use feel. Consumers preferred the product to this top benchmark COMP 1 due to the inventive formulation INV 1 clean look, pleasant skin feel (no desire to wash face afterwards), and gentleness on the face.

Both products were deemed by users to be effective, gentle on skin, and provide a quick removal process. Notably, while the overall performance of the inventive and comparative proved comparable, the key benefits of the inventive are a surprisingly effective removal in a water based composition that does not require rinsing after use and yet leaves the skin without a greasy, oily feel.

TABLE 16
User Qualitative Journey (key to FIG. 3)

| Experience | Time* | Label | Summary | INV 1 | COMP 1 |
|---|---|---|---|---|---|
| Appropriate | BEFORE | A | Product Scent -Both | COMPARABLE | COMPARABLE |
| scent | | | performed similar | | |
| Consistency | BEFORE | B | Product Consistency- | BETTER | MIXED |
| | | | INV 1 performed well | | |
| | | | with COMP 1r mixed | | |
| Easy to use | USAGE | C | Easy Application | COMPARABLE | COMPARABLE |
| | | | &AmountSprayed/ | | |
| | | | Coverage -Both performed | | |
| | | | similar | | |
| Satisfactory | USAGE | D | SEE ABOVE | COMPARABLE | COMPARABLE |
| amount | | | | | |
| sprayed | | | | | |
| Force of Spray | USAGE | E | Force of Spray -COMP 1 | NEUTRAL | BETTER |
| | | | performs well, while | | |
| | | | INV 1 is neutral | | |
| Spray | USAGE | F | | COMPARABLE | COMPARABLE |
| coverage | | | | | |
| Quick Removal | USAGE | G | Quick & Complete | COMPARABLE | COMPARABLE |
| of makeup and | | | Removal - Both | | |
| mascara | | | products performed | | |
| | | | similarly | | |
| Complete | USAGE | H | SEE ABOVE | COMPARABLE | COMPARABLE |
| Removal of | | | | | |
| makeup and | | | | | |
| mascara | | | | | |
| No rubbing | USAGE | I | No Rubbing - Both | UNDER | UNDER |
| | | | productsunder | PERFORMED | PERFORMED |
| | | | performed | | |
| Skin looked | USAGE | J | Clean look on skin -INV | GOOD | MIXED |
| clean | | | 1 performed well with | | |
| | | | COMP 1r mixed | | |
| Skin felt clean | USAGE | K | Skin feel - INV 1 | GOOD | POOR |
| | | | performs well for skin | | |
| | | | feel attributes. While | | |
| | | | COMP 1r is less | | |
| | | | appreciated for skin | | |
| | | | feelattributes with | | |
| | | | addition to needing to | | |
| | | | wash face after | | |
| | | | application that is | | |
| | | | unusual behavior | | |
| No unwanted | USAGE | L | SEE ABOVE | GOOD | POOR |
| residue | | | | | |
| No sticky | USAGE | M | SEE ABOVE | GOOD | POOR |
| residue | | | | | |
| No oily residue | USAGE | N | SEE ABOVE | GOOD | POOR |
| Gentle on skin | AFTER | O | Gentle - INV 1 | GOOD | NEUTRAL |
| eye | | | isfavorable for feeling | | |
| | | | gentle on skin/eye area. | | |
| | | | While COMP 1r is | | |
| | | | neutral | | |
| Needed to | AFTER | P | SEE ABOVE | GOOD | POOR |
| wash after | | | | | |
| (unusual) | | | | | |
| Pump | AFTER | Q | SEE ABOVE | GOOD | NEUTRAL |
| experience | | | | | |
| (spray on skin) | | | | | |

### Example 11: Clinical Study B: Mascara Removal; Evaluation of Removal Efficacy

COMP 1 and INV 1 were evaluated. Target N=18 volunteers Caucasian women; Ages 18-60, Medium-Long Lashes, No hooded eyelids Tested Products Maybelline Colossal Big Shot Very Black Waterproof Mascara.

Makeup was applied to all subjects in the same manner, according to a standardized procedure that includes prior washing. Mascara removal was accomplished by the same standardized procedure for all subjects.

References in this example to Inventive 1 include the same formulation in specific packaging (SP).

The difference in actuators made the experience very different for the volunteers. COMP 1 (aerosol spray) is a lightweight mist, which makes it seem like nothing is being applied to the face. At times, volunteers even questioned if there was anything on. INV 1 has a more wetting effect during application. The subjects could feel the product on the face that signals delivery to skin.

Dry hand emulsification is key to making the spray removers work their best. Immediately with the first rounds of circular motions around the lashes it is evident that the product breaks down. COMP 1 spray broke down a little bit quicker due to the amount of oil it has in it. Both products appear & and were reported to feel different on the skin. COMP 1 has an oily feel/look on the face and due to the wide mist, particles of oil are left around the hairline which makes the hair greasy (which may or may not be consumer acceptable). INV 1 has a very wetting look once applied, does break down easily during emulsification and has an overall easy removal, just very slight resistance on the lashes during removal with cotton rounds since there is not much slip to the product itself (Was not perceived by the consumer).

Overall, both makeup remover sprays perform in the high efficiency category when emulsifying with dry hands. COMP 1 performed slightly better than market leading COMP 2. INV 1 performed similar to the COMP 2 and COMP 3. Different methods of removal are used between Bi-Phase & spray galenics, however overall, both products are highly efficient waterproof mascara removers.

TABLE 17
Mascara Removal with Inventive vs. Comparatives

| Residue Check Score | | | | |
|---|---|---|---|---|
| 0-1.0 | 1.5-2.5 | 3.0-3.5 | 4.0-4.5 | 5.0-6.0 |

| | | | |
|---|---|---|---|
| INV 1 SP | Biphase | 1.0 | |
| | spray | | |
| COMP 1 | Biphase | 0.5 | |
| | spray | | |
| Comp 2 | Bi-phase | 1.0 | |
| Comp 3 | Bi-phase | 1.0 | |
| Comp 4 | Micellar | | 5.0 |
| | Water | | |

### Example 12: Clinical Study C: Mascara Removal; Evaluation of Removal Efficacy

Tests were conducted to demonstrate the performance of new packaging options for INV 1. Target N=6 volunteers Caucasian women; Ages 18-60, Medium-Long Lashes, No hooded eyelids Tested Products INV 1, INV 1 CP, and COMP 1-7 with Maybelline Colossal Big Shot Very Black Waterproof Mascara.

References in this example to Inventive 1 include the same formulation in different packaging (SP and CP).

N Makeup was applied to all subjects in the same manner, according to a standardized procedure that includes prior washing. Mascara removal was accomplished by the same standardized procedure for all subjects.

Conclusion Overall, INV 1 using similar sprayer to Inv 8 is quite lower in removal efficacy when compared to the original prototype makeup remover INV 1 CP. The original prototype is a High Efficiency stubborn mascara remover, however with the change pump INV 1, the efficiency decreases down to a Medium category. The formulas are the same, but the impact of how much product comes out to the targeted area is different. The INV 1 Pump sprays everywhere around the perimeter of the face but not close enough to the eye area to get proper saturation for removal, hence the lower efficacy results. Both competitor products Inv 5 and 6 makeup remover Spray perform as Med-High Efficiency stubborn mascara removers. Inv 8 is slightly more efficient than the Inv 7 with a 1 cotton round difference (median).

TABLE 18
Mascara Removal with Inventive in alternative
pump forms vs. Comparatives

| Residue Check Score | | | | |
|---|---|---|---|---|
| 0-1.0 | 1.5-2.5 | 3.0-3.5 | 4.0-4.5 | 5.0-6.0 |

| | | | | | |
|---|---|---|---|---|---|
| INV 1 SP | Biphase | 1.0 | | | |
| | Pump spray | | | | |
| INV 1 CP | Biphase spray | 1.0 | | | |
| | w/childproof | | | | |
| | pump | | | | |
| COMP 1 | Biphase | 0.5 | | | |
| | Pump spray | | | | |
| Comp 2 | Bi-phase | 1.0 | | | |
| Comp 3 | Bi-phase | 1.0 | | | |
| Comp 4 | Micellar Water | | | | 5.0 |
| Comp 5 | Aerosol Spray | | 1.5 | | |
| Comp 6 | Pump (non- | | 2.5 | | |
| | aerosol) Spray | | | | |
| Comp 7 | Pump (non- | | | 3.0 | |
| | aerosol) Spray | | | | |

### Example 13: Clinical Study D: Mascara Removal; Evaluation of Removal Efficacy

Tests were conducted to demonstrate the performance of new packaging options for INV 1 with different actuators (CP, HDM and OP). Target N=6 volunteers Caucasian women; Ages 18-60, Medium-Long Lashes, No hooded eyelids Tested Products INV 1, INV 1 CP, INV 1 HDM and INV1 OP, and COMP 1-7.

References in this example to Inventive 1 include the same formulation in different packaging (SP, CP, HDM and OM).

Makeup was applied to all subjects in the same manner, according to a standardized procedure that includes prior washing. Mascara removal was accomplished by the same standardized procedure for all subjects.

Overall, both makeup remover sprays perform in the high efficiency category when emulsifying with dry hands. COMP 1 performed slightly better than the leading market makeup remover Comp 2. The 2 tested formulas (INV 1 HDM & INV 1 OM) performed similar to the Bi-Phase products Comp 2 & Comp 3. Different methods of removal are used between Bi-Phase & spray galenic, however overall, both products are highly efficient waterproof mascara removers.

TABLE 19
Mascara Removal with Inventive vs. Comparatives (~0.2 g/spray)

| Application | Residue Check Score | | | | |
|---|---|---|---|---|---|
| Type | 0-1.0 | 1.5-2.5 | 3.0-3.5 | 4.0-4.5 | 5.0-6.0 |

| | | | | | |
|---|---|---|---|---|---|
| INV 1 SP | Biphase | 1.0 | | | |
| | Pump spray | | | | |
| INV 1 CP | Biphase spray | 1.0 | | | |
| | w/childproof | | | | |
| | pump | | | | |
| INV 1 | Biphase Pump | | | | |
| HDM | Misting Spray | | | | |
| | (droplet size | | | | |
| | 40-300 microns) | | | | |
| INV 1 | Biphase HD | | | | |
| OM | Pump - oil | | | | |
| | mister | | | | |
| COMP 1 | Biphase | 0.5 | | | |
| | Pump spray | | | | |
| Comp 2 | Bi-phase | 1.0 | | | |
| Comp 3 | Bi-phase | 1.0 | | | |
| Comp 4 | Micellar Water | | | | 5.0 |
| Comp 5 | Aerosol Spray | | 1.5 | | |
| Comp 6 | Pump (non- | | 2.5 | | |
| | aerosol) Spray | | | | |
| Comp 7 | Pump (non- | | | 3.0 | |
| | aerosol) Spray | | | | |

These results provide evidence that fine mist spray packaging produces the same category of high efficacy in a bi-phase composition with water and oil phases as competitors with 100% Oil Composition (COMP 1).

### Example 14: Clinical Study E: Evaluation of Inventive (INV 1) Mist Spray Pump Container Vs. Benchmark Aerosol (COMP 1) (Propellant Based) Spray

Study was undertaken to explore consumer perceptions and preference for the formula and package by assessing full product journey including application, texture, feel, end look, wear and removal.

Methodology: 14-day in-home use test was conducted; 42 female users ages 18-30 with a mix of ethnicities, skin types and tones and users of mass and lux brand makeups had 4 active days and tested the INV1 and COMP1 products via a randomized sequential monadic design across two consumer markets; the product use protocol was provided including specific instructions for product application, and makeup and mascara removal; subjects and provided responses to questions about the product application and sensorial experience.

The results are tabulated below in Table 20.

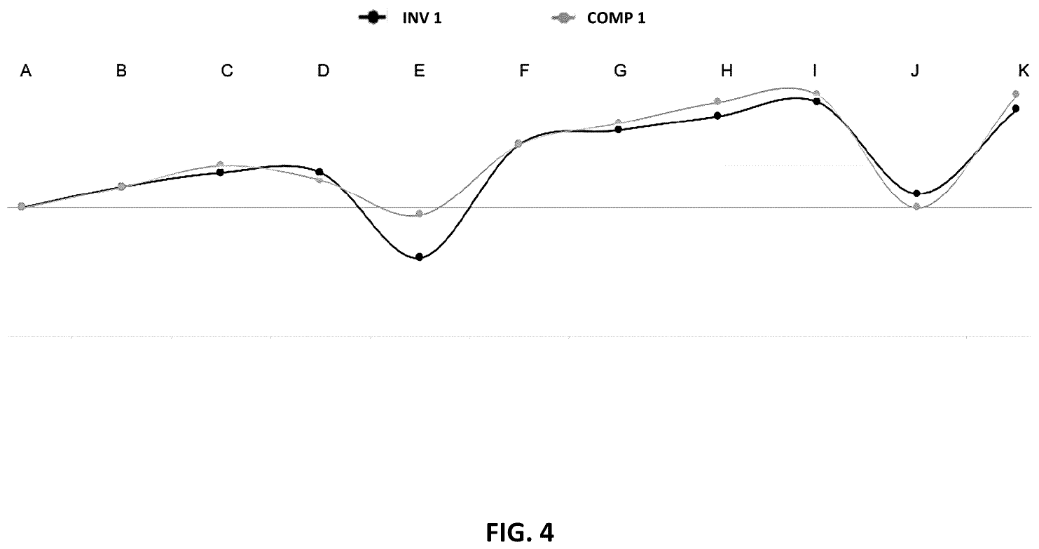

Tested: INV 1 vs COMP 1: Referring to FIG. 4, the chart details the journey of the consumer using the delivery mist pump for INV 1 and the aerosol spray for COMP 1.

Referring again to FIG. 4, noting that these compositions differ significantly in terms of the amount and nature of the oil content where COMP 1 is anhydrous, includes silicone oils/silicones and strong surfactants, INV 1 as delivered in the mist pump demonstrates an overall favorable user experience with both products demonstrating an opportunity to improve with respect to direct application onto the face, and initial feel of oiliness on the skin.

Both products were deemed by users to be effective, gentle on skin, and provide a quick removal process. Notably, the overall performance of the inventive and comparative delivery containers proved comparable, the key benefits of the inventive are the elimination of use of aerosol propellants and overall reduction of use of silicones and surfactants without compromising effectiveness.

TABLE 20
User Product Applicator Qualitative Journey

| | | | | | |
|---|---|---|---|---|---|
| Experience | Time | Label | Summary | INV 1 | COMP 1 |
| Visual | BEFORE | A | Test subjects are open | COMPARABLE | COMPARABLE |
| discovery | | | to the idea of a spray | | |
| | | | format and excited to | | |
| | | | trythe product. | | |
| Viewing | BEFORE | B | Instructions are clear | COMPARABLE | COMPARABLE |
| Instructions | | | and easy tounderstand, | | |
| | | | though many choose to | | |
| | | | ignore them after initial | | |
| | | | trial. | | |
| Handling | BEFORE | C | SEE ABOVE | | SLIGHT |
| | | | | | PREFERENCE |
| Shaking | BEFORE | D | The shaking is easy, and | SLIGHT | |
| | | | the instructions are | PREFERENCE | |
| | | | helpful in knowing how | | |
| | | | long it must be shaken. | | |
| Applying on | USAGE | E | Most don't like | LEAST | NOT FAVORED |
| Face | | | spraying directly | FAVORED | |
| | | | ontheir face, and the | | |
| | | | force of the optimized | | |
| | | | product feels too | | |
| | | | strong. Ultimately, | | |
| | | | many switch to | | |
| | | | spraying on a cotton | | |
| | | | pad or paper towel | | |
| | | | first. | | |
| Smelling | USAGE | F | Once on the skin, the | COMPARABLE | COMPARABLE |
| Product | | | smell isacceptable and | | |
| | | | it is easy to massage | | |
| | | | into the face. | | |
| Massaging | USAGE | G | SEE ABOVE | COMPARABLE | COMPARABLE |
| Face | | | | | |
| Makeup | USAGE | H | Users are impressed | COMPARABLE | COMPARABLE |
| Removal | | | with both products | | |
| | | | ability to remove even | | |
| | | | the toughest makeup - | | |
| | | | matte lip, waterproof | | |
| | | | mascara, etc. | | |
| Noticing | AFTER | I | Skin looks clean | UNDER | UNDER |
| instant look | | | andfree of makeup. | PERFORMED | PERFORMED |
| | | | Some feel they may not | | |
| | | | have to cleanse after, | | |
| | | | butmost likely still | | |
| | | | would. | | |
| Noticing | AFTER | J | Some experienced an | GOOD | MIXED |
| instant feel | | | oily or dry feeling on | | |
| | | | the skin immediately | | |
| | | | after applying, but after | | |
| | | | drying it usually | | |
| | | | neutralized. | | |
| Removal | AFTER | K | Test subjects see the | GOOD | POOR |
| Results | | | amount of makeup | | |
| | | | removed on their | | |
| | | | cotton rounds and are | | |
| | | | impressed. | | |

While the disclosure has been described with reference to a preferred embodiment, it will be understood by those skilled in the art that various changes may be made, and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising,” “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

In some embodiments, the terms “Exclude,” “Free” and “Essentially Free” mean that no reliably measurable excluded material, typically 0% by weight, based on the total weight of the makeup removal composition, or, for example, no amount greater than about 5%, by weight, based on the total weight of the makeup removal composition, of any excluded material as described herein, is present in the makeup removal composition. The terms “Exclude,” “Free” and “Essentially Free” mean that, while it is preferred that no excluded material is present in the makeup removal composition, it is possible to have very small amounts of the excluded material in the makeup removal composition of the invention, provided that these amounts do not materially affect the advantageous properties of the makeup removal composition. In particular, the terms “Exclude,” “Free” and “Essentially Free” mean that material can be present in the makeup removal composition at an amount of less than 5%, or less than 4%, or less than 3%, or less than 2%, or less than 1%, or less than 0.1% by weight, based on the total weight of the makeup removal composition. In accordance with the various embodiments, it is contemplated that some or all of following ingredients may be excluded from the makeup removal composition according to the invention: alcohols, for example but not limited to, monohydric alcohols; powders comprising Nylon-12, polymethyl methacrylate, polyethylene beads, and powders which are considered microplastics; lactic acid, glycolic acid, tartaric acid, mandelic acid, citric acid, salicylic acid and derivatives thereof (including 5-n-octanoylsalicylic acid, salicylate, sodium salicylate, and willow extract), capryloyl salicylic acid, beta hydroxybutanoic acid, propionic acid, beta-hydroxy beata-methylbutyric acid, carnitine tropic acid, and trethocanic acid; alkyl polyglucosides selected from the group consisting of decyl glucoside, lauryl glucoside, octyl glucoside, coco glucoside, caprylyl/capryl glucoside, and sodium lauryl glucose carboxylate, and combinations thereof; surfactants with a C10-C20 fatty alcohol or acid hydrophobe condensed with from about 2 to about 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan; mono- and di-C8-C20 fatty acids; polyoxyethylene sorbitan; alkyl polyglycosides and saccharide fatty amides (e.g. methyl gluconamides); alkyl ether sulfate and sulfonates; alkyl sulfates and sulfonates; alkylbenzene sulfonates; alkyl and dialkyl sulfosuccinates; C8-C20 acyl isethionates; C8-C20 alkyl ether phosphates; alkylethercarboxylate, PEG-100 Stearate; PEG-20 Stearate and other esters of Poly(Ethylene Glycol); polyethylene glycol (PEG) surfactants, polypropylene glycol (PPG) surfactants, sulfate surfactants; taurates, for example, taurate surfactants; Sucrose Stearate and other emulsifiers based on sugar esters; Glyceryl Stearate and other glycerol esters; Disodium Ethylene Dicocamide PEG-15 Disulfate; Sodium Steroyl Glutamate and other fatty amides; Steareth-100 and other fatty ethers, cationic surfactants, and other cationic compounds; silicones, such as silicone polymers, silicone elastomers, and silicone oils, for example selected from dimethicone, cyclopentasiloxane, cyclohexasiloxane, and combinations thereof; phthalates, parabens, sulfates, polyquaternium, microplastics, synthetic dyes, gelling agents, and combinations thereof.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

What is claimed is:

1. A makeup a makeup removal composition, comprising: a bi-phase micellar system comprising an oil phase and a water phase, the system comprising:
a. a blend of oils comprising
iii. one or more branched or linear liquid alkanes with carbon chain length of C11 to C20, comprising isohexadecane and isododecane, and
iv. one or more emollient esters;
b. at least one polymeric thickener for a water-based system;
c. at least one polyglyceryl nonionic surfactant; and
d. water present from about 30% to about 70% by weight of the makeup removal composition
wherein the composition is a micellar bi-phase wherein water and oil are present in a ratio of water to oil from about 1.5:1 to about 1:1.5.

2. The makeup removal composition according to claim 1, formulated as an aesthetically pleasing two-phase formulation with a phase interface and which allows easy make-up removal that is not greasy, and includes little or no components that may be irritating to the skin or eyes.

3. The makeup removal composition according to claim 1, wherein the makeup removal composition is essentially free of silicone.

4. The makeup removal composition according to claim 1, wherein the composition excludes silicones and one or more of alcohols, sulfate-containing compounds, PEG surfactants, xanthan gum, or combinations thereof.

5. The makeup removal composition according to claim 1, wherein the makeup removal composition comprises one or more additives including preservatives, fragrances, actives, and pH adjusters, among other cosmetically acceptable additives.

6. The makeup removal composition according to claim 1, wherein the one or more emollient esters comprises isopropyl myristate, and the at least one polymeric thickener for a water-based system comprises hydroxyethylcellulose.

7. The makeup removal composition according to claim 1, wherein isohexadecane is present from about 15% to about 30% and isododecane is present from about 10% to about 20%, each by weight, based on the weight of the makeup removal composition.

8. The makeup removal composition according to claim 1, wherein isohexadecane is present at about 28% and isododecane is present at about 15%, each by weight, based on the weight of the makeup removal composition.

9. The makeup removal composition according to claim 1, wherein the one or more emollient esters is present from about 1% to about 8% by weight, based on the weight of the makeup removal composition.

10. The makeup removal composition according to claim 1, wherein the one or more emollient esters comprises isopropyl myristate present from about 1% to about 8% by weight, based on the weight of the makeup removal composition.

11. The makeup removal composition according to claim 1, wherein the total amount of emollient ester does not exceed a total of about 10% by weight of the makeup removal composition.

12. The makeup removal composition according to claim 1, wherein the at least one polymeric thickener for a water-based system is present from about 0.01% to about 1.0% by weight, based on the weight of the makeup removal composition.

13. The makeup removal composition according to claim 1, wherein, the at least one polymeric thickener for a water-based system comprises hydroxyethylcellulose and wherein the at least one polyglyceryl nonionic surfactant comprises Polyglyceryl-6 Caprylate and Polyglyceryl Dicaprate.

14. The makeup removal composition according to claim 1, wherein the at least one polyglyceryl nonionic surfactant is present in a total amount from about 0.01% to about 0.2%, by weight, based on the weight of the composition.

15. The makeup removal composition according to claim 1, wherein the at least one polyglyceryl nonionic surfactant comprises polyglyceryl-6 Caprylate present at about 0.025% and Polyglyceryl Dicaprate present at about 0.025%, each by weight, based on the weight of the composition.

16. The makeup removal composition according to claim 1, wherein the makeup removal composition comprises triethyl citrate.

17. The makeup removal composition according to claim 1, wherein the makeup removal composition includes one or a combination of additives selected from sodium hydroxide, panthenol, hydroxyacetophenone, gluconolactone, myrtrimonium bromide, sodium citrate, sodium chloride, dicaprylyl ether, citric acid, or combinations thereof.

18. A makeup removal composition, comprising: a bi-phase micellar system comprising an oil phase and a water phase, the system comprising:
a. a blend of oils comprising
i. one or more branched or linear liquid alkanes with carbon chain length of C11 to C20, comprising isohexadecane present at about 28%, and isododecane present at about 15%, and
ii. one or more emollient esters comprising isopropyl myristate present at about 1%;
b. at least one polymeric thickener for a water-based system comprising hydroxyethylcellulose present at about 0.025%;
c. at least one polyglyceryl nonionic surfactant comprising Polyglyceryl-6 Caprylate present at about 0.025% and Polyglyceryl Dicaprate present at about 0.025%;
d. water present from about 45% to about 55%;
e. triethyl citrate; and
f. one or a combination of additives selected from sodium hydroxide, panthenol, hydroxyacetophenone, gluconolactone, myrtrimonium bromide, sodium citrate, sodium chloride, dicaprylyl ether, citric acid, or combinations thereof, all amounts by weight, based on the weight of the makeup removal composition
wherein the composition is a micellar bi-phase wherein water and oil are present in a ratio of water to oil of about 1:1

19. A product comprising: a container containing the makeup removal composition according to claim 1; an outlet connected to the container, such as an outlet of a spray nozzle; and a spray-device, wherein the spray-device is capable of discharging the makeup removal composition in the container via the outlet, wherein the spray nozzle is a fine mist nozzle that delivers a mist having a pump volume in a range from about 0.01 to about 1.0 ml/pump, and producing mist particles of micron scale in a range from about 10 microns to about 900 microns.

20. The product according to claim 19, wherein the makeup removal composition may be applied for use and removed by wiping without rinsing with water.
